(19) United States
(12) Patent Application Publication
(10) Pub. No.: US 2018/0368967 A1
(43) Pub. Date: Dec. 27, 2018

(54) ARTIFICIAL BLOOD VESSEL AND METHOD FOR MAKING THE SAME

(71) Applicant: HON HAI PRECISION INDUSTRY CO., LTD., New Taipei (TW)

(72) Inventor: HSIU-WEN CHIEN, New Taipei (TW)

(21) Appl. No.: 15/638,827

(22) Filed: Jun. 30, 2017

(30) Foreign Application Priority Data
Jun. 23, 2017 (TW) 106121032

(51) Int. Cl. A61F 2/06; D01D 5/00; A61L 27/50

(52) U.S. Cl. CPC A61F 2/062; A61F 2/06; A61L 27/507; D01D 5/0076

(57) ABSTRACT

An artificial blood vessel includes a nanofiber base film and a nanofiber external film connected to the nanofiber base film. The nanofiber base film comprises a plurality of polymer nanofibers aligned according to a first single-direction aligning pattern. The nanofiber external film comprises a plurality of polymer nanofibers aligned according to a second aligning pattern that is perpendicularly different from the first aligning pattern.

### FIELD

The subject matter herein generally relates to medical technology, and more particularly, to an artificial blood vessel and a method for making the artificial blood vessel.

### BACKGROUND

Intravascular stents are generally tubular prosthesis that can be placed within a body passageway such as any vein, artery, or blood vessel within the vascular system. The intravascular stent usually contains endothelial cells. However, seeding the endothelial cells on the tubular intravascular stent is problematic.

### BRIEF DESCRIPTION OF THE DRAWINGS

Implementations of the present technology will now be described, by way of example only, with reference to the attached figures.

FIG. 1 is a flowchart of an exemplary embodiment of a method for making an artificial blood vessel.

FIG. 2 is a diagram of an electrospinning device used in the method in FIG. 1.

FIG. 3 is a diagram of a nanofiber base film made by the electrospinning device of FIG. 2.

FIG. 4 is a diagram showing a nanofiber deformable film formed on the nanofiber base film of FIG. 3, to form a nanofiber composite film.

FIG. 5 is a diagram showing the nanofiber composite film of FIG. 4 under ultraviolet radiation, to form the artificial blood vessel.

FIG. 6 illustrates a reaction in the nanofiber composite film of FIG. 4, when under ultraviolet radiation.

FIG. 7 is a diagram showing the alignment of the nanofiber base film and the nanofiber deformable film of nanofiber composite film of FIG. 4.

FIG. 8 is similar to FIG. 7, but showing the nanofiber base film and the nanofiber deformable film being aligned in a different way.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, it will be understood by those of ordinary skill in the art that the embodiments described herein can be practiced without these specific details. In other instances, methods, procedures, and components have not been described in detail so as not to obscure the related relevant feature being described. Also, the description is not to be considered as limiting the scope of the embodiments described herein. The drawings are not necessarily to scale, and the proportions of certain parts may be exaggerated to better illustrate details and features of the present disclosure.

The disclosure is illustrated by way of example and not by way of limitation in the figures of the accompanying drawings, in which like references indicate similar elements. It should be noted that references to “an” or “one” embodiment in this disclosure are not necessarily to the same embodiment, and such references mean at least one.

The term “comprising,” when utilized, means “including, but not necessarily limited to”; it specifically indicates open-ended inclusion or membership in the so-described combination, group, series, and the like.

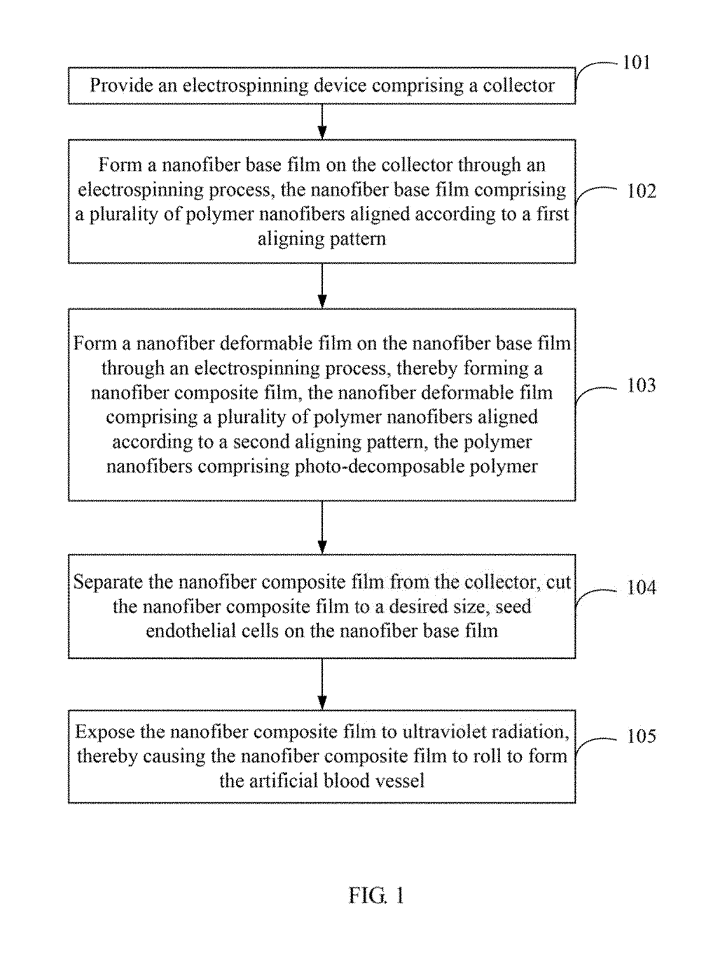

FIG. 1 illustrates a flowchart of an embodiment for a method for making an artificial blood vessel. The exemplary method is provided by way of example, as there are a variety of ways to carry out the method. Each block shown in the figure represents one or more processes, methods, or subroutines, carried out in the exemplary method. Furthermore, the illustrated order of blocks is by example only, and the order of the blocks can change. Additional blocks may be added or fewer blocks may be utilized, without departing from this disclosure. The exemplary method can begin at block 101.

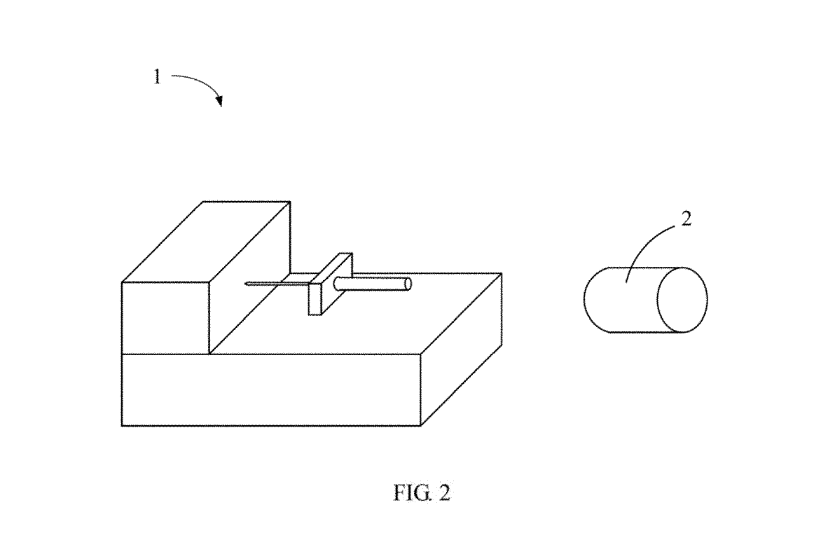

At block 101, referring to FIG. 2, an electrospinning device 1 is provided that comprises a collector 2.

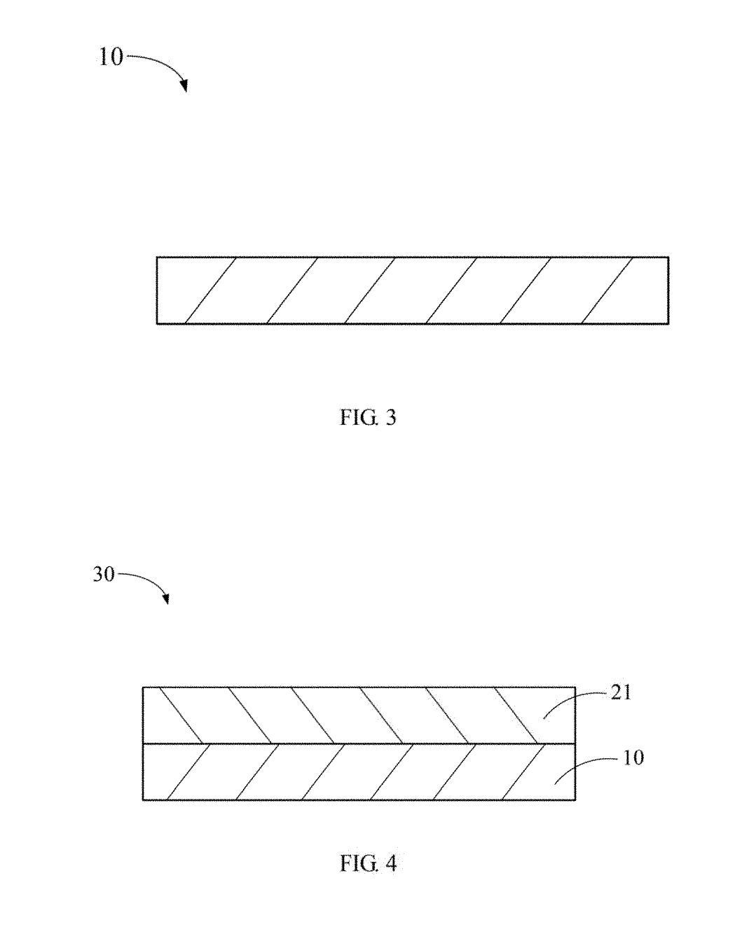

At block 102, referring to FIG. 3, a nanofiber base film 10 is formed on the collector 2 through an electrospinning process. The nanofiber base film 10 comprises a number of polymer nanofibers aligned according to a first aligning pattern. The first aligning pattern is that the polymer fibers of the nanofiber base film 10 are orderly aligned along a same direction.

In at least one exemplary embodiment, the polymer nanofibers of the nanofiber base film 10 comprise polycaprolactone (PCL) nanofibers and polyurethane (PU) nanofibers mixed together. The PCL nanofibers can provide biodegradability, and the PU nanofibers can provide a high flexibility.

The nanofiber base film 10 can be formed by an electrospinning solution comprising PCL, PU, and a solvent. The solvent can be selected from a group consisting of formic acid, acetic acid, acetone, dimethylformamide, dimethylacetamide, etrahydrofuran, dimethyl sulfoxide, hexafluoroisopropanol, trifluoroethanol, dichloromethane, trichlormethane, methanol, ethanol, chlorotoluene, dioxane, trifluoroethane, trifluoroacetic acid, water, and any combination thereof.

At block 103, referring to FIG. 4, a nanofiber deformable film 21 is formed on a surface of the nanofiber base film 10 facing away from the collector 2 through an electrospinning process, thereby forming a nanofiber composite film 30. The nanofiber deformable film 21 comprises a number of polymer nanofibers aligned according to a second aligning pattern that is different from the first aligning pattern.

In at least one exemplary embodiment, the polymer nanofibers of the nanofiber deformable film 21 comprise photo-decomposable polymer. In at least one exemplary embodiment, the polymer nanofibers of the nanofiber deformable film 21 comprise coumarin-containing PCL nanofibers and coumarin-containing PU nanofibers mixed together. The coumarin has a chemical structure diagram of

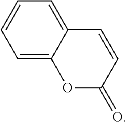 [SMILES: O=c1ccc2ccccc2o1]

The coumarin-containing PCL nanofibers and the coumarin-containing PU nanofibers have a chemical structure diagram of

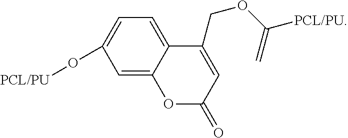

The nanofiber deformable film 21 can be formed by an electrospinning solution comprising coumarin-containing PCL, coumarin-containing PU, and a solvent. The solvent can be selected from a group consisting of formic acid, acetic acid, acetone, dimethylformamide, dimethylacetamide, etrahydrofuran, dimethyl sulfoxide, hexafluoroisopropanol, trifluoroethanol, dichloromethane, trichlormethane, methanol, ethanol, chlorotoluene, dioxane, trifluoroethane, trifluoroacetic acid, water, and any combination thereof.

At block 104, the nanofiber composite film 30 is separated from the collector 2 and cut to a desired size. Endothelial cells can be seeded on the nanofiber base film 10.

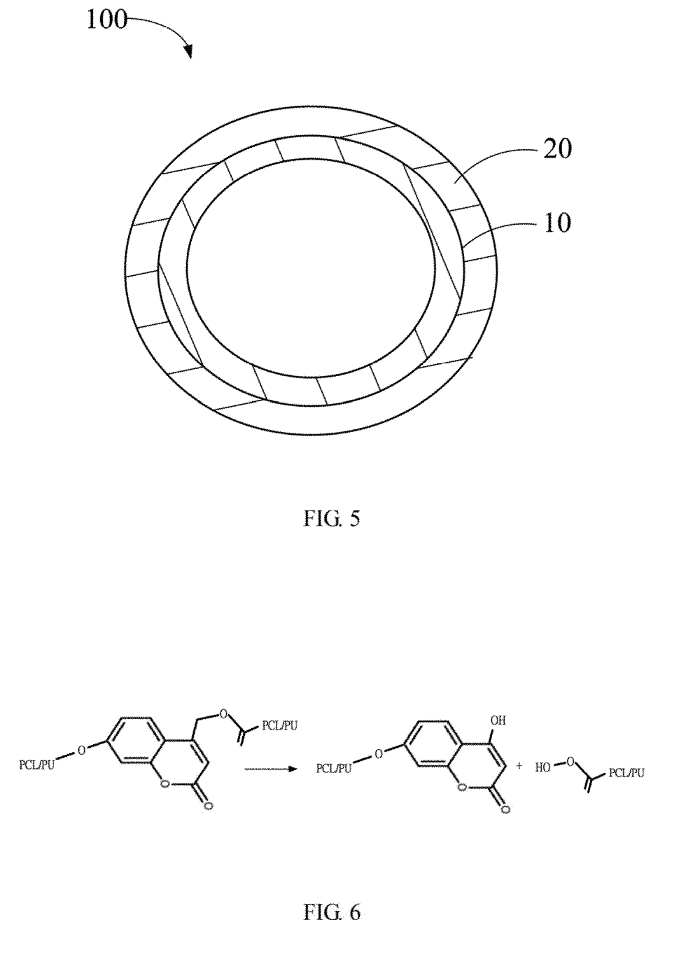

At block 105, the nanofiber composite film 30 after being cut is exposed to ultraviolet radiation. Thereby, the coumarin decomposes as shown in FIG. 6 to cause the nanofiber deformable film 21 to expand and roll to form a nanofiber external film 20 (FIG. 5). That is, the nanofiber composite film 30 in FIG. 4 rolls to form the artificial blood vessel 100.

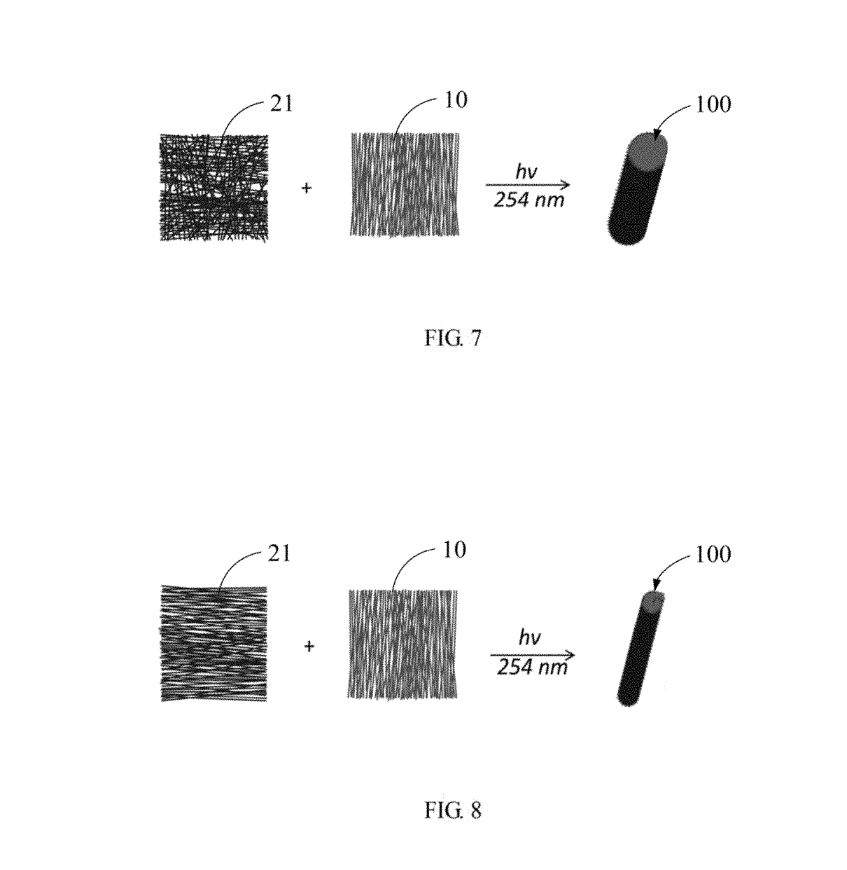

Referring to FIG. 7, in at least one exemplary embodiment, the first aligning pattern is that the polymer fibers of the nanofiber base film 10 are orderly aligned along a same direction. The second aligning pattern is that the polymer fibers of the nanofiber deformable film 21 are randomly aligned.

Referring to FIG. 8, in another exemplary embodiment, the first aligning pattern is that the polymer fibers of the nanofiber base film 10 are orderly aligned along a same direction (first direction). The second aligning pattern is that the polymer fibers of the nanofiber deformable film 21 are orderly aligned along a second direction that is perpendicular to the first direction. The aligning pattern of the polymer nanofibers can be controlled by adjusting the rotating speed of the collector 2. For example, the polymer nanofibers are randomly aligned when the rotating speed of the collector 2 is 100 rpm. The polymer nanofibers are orderly aligned when the rotating speed of the collector 2 is 1500 rpm. Furthermore, the thicknesses of the nanofiber base film 10 and the nanofiber deformable film 21 can be controlled by adjusting the collecting time period of the collector 2.

The electrospinning process can be used to precisely control the aligning direction of the polymer nanofibers of the nanofiber base film 10 and the nanofiber deformable film 21. Furthermore, when the polymer nanofibers of the nanofiber base film 10 are controlled for alignment along the same direction, the rolling direction of the nanofiber deformable film 21 can be controlled. Also, the desired degree of rolling the nanofiber deformable film 21 (that is, the desired degree of rolling the artificial blood vessel 100) is affected by the aligning pattern of the polymer nanofibers of the nanofiber deformable film 21. In detail, when the nanofiber deformable film 21 expands and rolls under the ultraviolet radiation, the edges of the nanofiber deformable film 21 that are parallel to the aligning direction of the polymer nanofibers of the nanofiber base film 10 are resistant to rolling. The edges of the nanofiber deformable film 21 that are perpendicular to the aligning direction of the polymer nanofibers of the nanofiber base film 10 are less resistant to rolling. That is, the rolling direction of the nanofiber deformable film 21 should be perpendicular to the aligning direction of the polymer nanofibers of the nanofiber base film 10.

Moreover, since the polymer nanofibers of the nanofiber base film 10 are aligned along the same direction, the artificial blood vessel 100 also extends along the same direction. When endothelial cells are seeded on the nanofiber base film 10, the extending direction of the endothelial cells is also affected by the same direction, so that the endothelial cells can extend along the same direction. That is, the extending direction of the endothelial cells is the same as the direction of blood flow when the artificial blood vessel 100 is in use.

In at least one exemplary embodiment, a surface of the nanofiber deformable film 21 facing away from the nanofiber base film 10 is exposed to the ultraviolet radiation, thereby avoiding a decrease in activity of the endothelial cells under the ultraviolet radiation.

### Example 1

A nanofiber base film 10 was formed when the rotating speed of the collector 2 was 1500 rpm and the collecting time period of the collector 2 was 1 h. The nanofiber base film 10 comprised polymer nanofibers orderly aligned along a same direction, and had a thickness of 34 μm. A nanofiber deformable film 21 was formed on the nanofiber base film 10 to form a nanofiber composite film 30 when the rotating speed of the collector 2 was 100 rpm and the collecting time period of the collector 2 was 1.3 h. The nanofiber base film 10 comprised polymer nanofibers randomly aligned. The nanofiber composite film 30 had a total thickness of 84 The nanofiber composite film 30 was separated from the collector 2 and cut to 20×1.5 cm², and was then exposed to ultraviolet radiation of 254 nm for 1 second. An artificial blood vessel 100 was formed that had a diameter of 5 mm and a length of 20 cm.

### Example 2

A nanofiber base film 10 was formed when the rotating speed of the collector 2 was 1500 rpm and the collecting time period of the collector 2 was 1 h. The nanofiber base film 10 comprised polymer nanofibers orderly aligned along a first direction, and had a thickness of 34 The collector 2 was rotated about 90 degrees. A nanofiber deformable film 21 was formed on the nanofiber base film 10 to form a nanofiber composite film 30 when the rotating speed of the collector 2 was 1500 rpm and the collecting time period of the collector 2 was 1.3 h. The nanofiber base film 10 comprised polymer nanofibers orderly aligned along a second direction perpendicular to the first direction. The nanofiber composite film 30 had a total thickness of 78 The nanofiber composite film 30 was separated from the collector 2 and cut to 15×1.5 cm², and was then exposed to ultraviolet radiation of 254 nm for 0.5 second. An artificial blood vessel 100 was formed that had a diameter of 3 mm and a length of 15 cm.

### Example 3

A nanofiber composite film 30 was made according to the above EXAMPLE 2. Endothelial cells were seeded on the nanofiber base film 10. The nanofiber composite film 30 was then exposed to ultraviolet radiation of 254 nm for 0.5 second. An artificial blood vessel 100 was formed that had a diameter of 5 mm and a length of 20 cm.

FIG. 5 illustrates an exemplary embodiment of an artificial blood vessel 100. The artificial blood vessel 100 comprises a nanofiber base film 10 and a nanofiber external film 20 connected to the nanofiber base film 10. The nanofiber base film 10 is positioned at an inner side of the artificial blood vessel 100. The nanofiber external film 20 is positioned at an outer side of the artificial blood vessel 100.

The nanofiber base film 10 comprises a number of polymer nanofibers aligned according to a first aligning pattern. The first aligning pattern is that the polymer fibers of the nanofiber base film 10 are orderly aligned along a same direction. In at least one exemplary embodiment, the polymer nanofibers of the nanofiber base film 10 comprise PCL nanofibers and PU nanofibers mixed together. Endothelial cells can be seeded on the nanofiber base film 10.

The nanofiber external film 20 comprises a number of polymer nanofibers aligned according to a second aligning pattern that is different from the first aligning pattern. The polymer nanofibers of the nanofiber external film 20 comprise PCL nanofibers and PU nanofibers mixed together.

With the above configuration, since the photo-decomposable polymer can be decomposed under the ultraviolet radiation, the nanofiber deformable film 21 can expand and be rolled to form the artificial blood vessel 100, to obtain a desired shape and a desired degree of rolling. Thus, the endothelial cells can be seeded before the nanofiber deformable film 21 is rolled, thereby improving the operability for seeding the endothelial cells, comparing to seeding the endothelial cells on a tubular intravascular stent. Furthermore, since the polymer nanofibers of the nanofiber base film 10 are aligned along the same direction, when endothelial cells are seeded on the nanofiber base film 10, the endothelial cells can extend along the same direction. Moreover, the nanofiber composite film 30 can be cut according to a desired size of the artificial blood vessel 100. Thus, the size of the artificial blood vessel 100 can be precisely controlled to satisfy different users.

Depending on the embodiment, certain of the steps of methods described may be removed, others may be added, and the sequence of steps may be altered. It is also to be understood that the description and the claims drawn to a method may include some indication in reference to certain steps. However, the indication used is only to be viewed for identification purposes and not as a suggestion as to an order for the steps.

It is to be understood, even though information and advantages of the present embodiments have been set forth in the foregoing description, together with details of the structures and functions of the present embodiments, the disclosure is illustrative only; changes may be made in detail, especially in matters of shape, size, and arrangement of parts within the principles of the present embodiments to the full extent indicated by the plain meaning of the terms in which the appended claims are expressed.

What is claimed is:

1. A method for making an artificial blood vessel comprising:
providing an electrospinning device comprising a collector;
forming a nanofiber base film on the collector through an electrospinning process, the nanofiber base film comprising a plurality of polymer nanofibers aligned according to a first aligning pattern, wherein the first aligning pattern is that the polymer fibers of the nanofiber base film are orderly aligned along a same direction;
forming a nanofiber deformable film on a surface of the nanofiber base film facing away from the collector through an electrospinning process, thereby forming a nanofiber composite film, the nanofiber deformable film comprising a plurality of polymer nanofibers aligned according to a second aligning pattern that is different from the first aligning pattern, the polymer nanofibers of the nanofiber deformable film comprising photo-decomposable polymer;
separating the nanofiber composite film from the collector and cutting the nanofiber composite film to a desired size; and
exposing the nanofiber composite film after being cut to ultraviolet radiation, so that the photo-decomposable polymer decomposes to cause the nanofiber deformable film to expand and roll, thereby causing the nanofiber composite film to roll to form the artificial blood vessel.

2. The method of claim 1, wherein the polymer nanofibers of the nanofiber base film comprise polycaprolactone nanofibers and polyurethane nanofibers mixed together.

3. The method of claim 2, wherein the nanofiber base film is formed by an electrospinning solution comprising polycaprolactone, polyurethane, and a solvent.

4. The method of claim 3, wherein the solvent is selected from a group consisting of formic acid, acetic acid, acetone, dimethylformamide, dimethylacetamide, etrahydrofuran, dimethyl sulfoxide, hexafluoroisopropanol, trifluoroethanol, dichloromethane, trichlormethane, methanol, ethanol, chlorotoluene, dioxane, trifluoroethane, trifluoroacetic acid, water, and any combination thereof.

5. The method of claim 1, wherein the photo-decomposable polymer is coumarin, and the polymer nanofibers of the nanofiber deformable film comprise coumarin-containing polycaprolactone nanofibers and coumarin-containing polyurethane nanofibers mixed together.

6. The method of claim 5, wherein the nanofiber deformable film is formed by an electrospinning solution comprising coumarin-containing polycaprolactone, coumarin-containing polyurethane, and a solvent.

7. The method of claim 6, wherein The solvent is selected from a group consisting of formic acid, acetic acid, acetone, dimethylformamide, dimethylacetamide, etrahydrofuran, dimethyl sulfoxide, hexafluoroisopropanol, trifluoroethanol, dichloromethane, trichlormethane, methanol, ethanol, chlorotoluene, dioxane, trifluoroethane, trifluoroacetic acid, water, and any combination thereof.

8. The method of claim 1, wherein before the step of exposing the nanofiber composite film after being cut to ultraviolet radiation further comprises:
seeding endothelial cells on the nanofiber base film.

9. The method of claim 8, wherein a surface of the nanofiber deformable film facing away from the nanofiber base film is exposed under the ultraviolet radiation.

10. The method of claim 1, wherein the second aligning pattern is that the polymer fibers of the nanofiber deformable film are randomly aligned.

11. The method of claim 1, wherein the first aligning pattern is that the polymer fibers of the nanofiber base film are orderly aligned along a first direction, and the second aligning pattern is that the polymer fibers of the nanofiber deformable film are orderly aligned along a second direction that is perpendicular to the first direction.

12. An artificial blood vessel comprising:
a nanofiber base film positioned at an inner side of the artificial blood vessel; and
a nanofiber external film positioned at an outer side of the artificial blood vessel and connected to the nanofiber base film;
wherein the nanofiber base film comprises a plurality of polymer nanofibers aligned according to a first aligning pattern, the first aligning pattern is that the polymer fibers of the nanofiber base film are orderly aligned along a same direction, the nanofiber external film comprises a plurality of polymer nanofibers aligned according to a second aligning pattern that is different from the first aligning pattern.

13. The artificial blood vessel of claim 12, wherein the polymer nanofibers of the nanofiber base film comprise polycaprolactone nanofibers and polyurethane nanofibers mixed together.

14. The artificial blood vessel of claim 12, wherein the polymer nanofibers of the nanofiber external film comprise polycaprolactone nanofibers and polyurethane nanofibers mixed together.

15. The artificial blood vessel of claim 12, wherein endothelial cells are seeded on the nanofiber base film.

16. A method for making an artificial blood vessel comprising:
forming a nanofiber base film, the nanofiber base film comprising a plurality of polymer nanofibers aligned according to a first aligning pattern, wherein the first aligning pattern is that the polymer fibers of the nanofiber base film are orderly aligned along a same direction;
forming a nanofiber deformable film on the nanofiber base film, thereby forming a nanofiber composite film, the nanofiber deformable film comprising a plurality of polymer nanofibers aligned according to a second aligning pattern that is different from the first aligning pattern, the polymer nanofibers of the nanofiber deformable film comprising photo-decomposable polymer;
cutting the nanofiber composite film to a desired size; and
exposing the nanofiber composite film after being cut to ultraviolet radiation, so that the photo-decomposable polymer decomposes to cause the nanofiber deformable film to expand and roll, thereby causing the nanofiber composite film to roll to form the artificial blood vessel.
